(19) United States
(12) Patent Application Publication
(10) Pub. No.: US 2025/0169778 A1
(43) Pub. Date: May 29, 2025

(54) MODULAR MEDICAL IMAGING SYSTEM

(71) Applicants: THALES, MEUDON (FR); BA HEALTHCARE, PACE (FR)

(72) Inventors: Stefania NUZZO, VELIZY-VILLACOUBLAY (FR); Guillaume ROYER, VELIZY-VILLACOUBLAY (FR); Jean-Baptiste GUIRIMAND, VELIZY-VILLACOUBLAY (FR); Nicolas COUSTAUD, VELIZY-VILLACOUBLAY (FR); David POTTIER-VINCENT, PACE (FR); François RIEFFEL, PACE (FR)

(21) Appl. No.: 18/954,377

(22) Filed: Nov. 20, 2024

(30) Foreign Application Priority Data
Nov. 23, 2023 (FR) 2312966

(51) Int. Cl. A61B 6/00

(52) U.S. Cl. CPC A61B 6/4411; A61B 6/4405; A61B 6/54; A61B 6/56

(57) ABSTRACT

A medical imaging system including a support having a frame and at least two wheels assembled directly or indirectly to the frame so as to allow the support to be moved; a medical imaging device; a control device configured to control the medical imaging device; and an electric power supply device configured to supply electrical energy to the medical imaging device and/or the control device, the medical imaging device, the control device and the electric power supply device each being configured to be removably assembled to the support.

### CROSS-REFERENCE TO RELATED APPLICATION

This application claims priority to foreign French patent application No. FR 2312966, filed on Nov. 23, 2023, the disclosure of which is incorporated by reference in its entirety.

### FIELD OF THE INVENTION

The invention relates to a medical imaging system.

In the medical field, it may be difficult or even impossible for a patient to get to the tools or systems that allow medical imaging to be performed. There are therefore medical imaging systems of the prior art that allow a medical professional to come to the patient in order to perform the medical imaging.

### BACKGROUND

In this context, systems such as the one described in the document EP3064140 are known. These are medical imaging systems equipped with wheels that allow them to be moved over short distances (typically from room to room within a hospital).

Systems of this type are heavy, bulky and difficult to transport. For these reasons, these systems are used only at one location where they may be moved about, in particular in a hospital/medical environment, but they are not moved or transported outside that location.

There are also other transportable medical imaging systems that are less heavy and that can be transported to different locations. However, these systems are either still so heavy and bulky as to require the use of a large vehicle such as a van to transport them, or, as regards the lightest systems, are insufficiently powerful to allow more than just a limited number of examinations (radiography of one limb only, etc.).

### SUMMARY OF THE INVENTION

The aim of the invention is to make available a medical imaging system that is both easy to transport and powerful enough to enable a wide range of medical examinations to be carried out.

To this end, the invention proposes a medical imaging system comprising:
a support having a frame;
at least two wheels assembled directly or indirectly to the frame so as to allow said support to be moved;
a medical imaging device;
a control device configured to control the medical imaging device; and
an electric power supply device configured to supply electrical energy to the medical imaging device and/or the control device,
   the medical imaging device, the control device and the electric power supply device each being configured to be removably assembled to the support,
   the support further comprising two longitudinal members assembled to the frame and configured to pivot relative to the frame between an operational position and a stowage position, the medical imaging system further comprising an X-ray source, an X-ray detector and a case, the case being dimensioned to house the X-ray source and/or the X-ray detector, the case having a width substantially equal to a distance between the longitudinal members.

The terms “assembly” and in particular “removable assembly” are understood to mean the fact that elements are connected or joined together in a detachable manner. This assembly can be, for example, a mechanical assembly (such as detachable fixing, elastic interlocking, clipping, arranging in a seat or on a receiving surface) or a magnetic assembly. By virtue of removable assembly, the medical imaging system according to the invention is modular. This facilitates transport since the different parts of the medical imaging system can be manipulated and stowed separately, for example in a vehicle for transport. The medical imaging system configured in this way can typically be transported in a standard trunk of a car (in particular of the kind in a compact car or sedan).

By virtue of the modular nature of the medical imaging system, there is no need for concessions as regards its electric power, since a powerful battery can be used.

The modular nature of the medical imaging system also makes it possible, in the event of a fault for example, to replace or repair a single part or module of the system and to intervene easily for its replacement or repair.

The structure of the support with wheels also makes it easier to move and transport the medical imaging system. The support carrying the other parts of the medical imaging system (the medical imaging device, the control device and the electric power supply device) can be pulled or pushed.

The medical imaging system is advantageously compact and of light weight. Preferably, the medical imaging system weighs less than 60 kilograms.

Some preferred and particularly convenient features of the medical imaging system according to the invention are presented below.

The medical imaging system can further comprise a housing configured to be removably assembled to the support.

The wheels of the medical imaging system can be removably assembled to the frame.

The wheels of the medical imaging system can be assembled to a base, said base being removably assembled to the frame.

The frame has two uprights and a gripping portion connecting the uprights.

The longitudinal members, in the operational position, can extend substantially horizontally, the longitudinal members, in the stowage position, then being parallel to the uprights of the frame and extending between said uprights.

Each longitudinal member can comprise a wheel at a free end.

The support can further comprise a receiving plate configured to receive the electric power supply device, the receiving plate being assembled to the frame and configured to pivot relative to the frame between an operational position and a stowage position, the receiving plate, in the operational position, extending substantially horizontally, the receiving plate, in the stowage position, being orthogonal to the uprights of the frame and extending substantially vertically.

The receiving plate can comprise at least one wheel on an underside.

The X-ray source can have an upper tube load limit of greater than or equal to 40 mAs.

The support can comprise a housing configured to receive the X-ray detector.

The support can comprise an arm assembled to the frame and having a free end configured to receive the X-ray source.

The arm can comprise a front section and a rear section articulated with respect to each other, the arm being movable between a service position and a retracted position, the front section and the rear section together forming a non-zero angle, preferably greater than 30°, when the arm is in the service position, the front section and the rear section being folded onto each other when the arm is in the retracted position.

The medical imaging system can further comprise an actuator assembled to the arm and configured to move the arm between the service position and the retracted position, the actuator preferably being an electric actuator.

### BRIEF DESCRIPTION OF THE DRAWINGS

Other features and advantages of the invention will become more apparent from the following description with reference to the attached drawings, which are given by way of non-limiting example:

FIG. 1 shows part of a medical imaging system according to an embodiment of the invention;

FIG. 2 shows a support of the medical imaging system in a first configuration;

FIG. 3 shows the support in a second configuration or stowage configuration;

FIG. 4 shows the medical imaging system in a third configuration or movement configuration;

FIGS. 5 and 6 show the medical imaging system in the course of being deployed from the stowage configuration; and

FIG. 7 shows the medical imaging system in a fourth configuration or deployment configuration.

### DETAILED DESCRIPTION

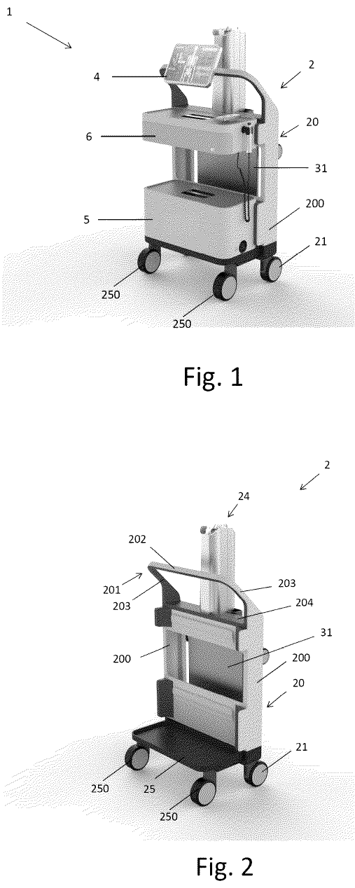

FIG. 1 shows part of a medical imaging system 1 according to an embodiment of the invention.

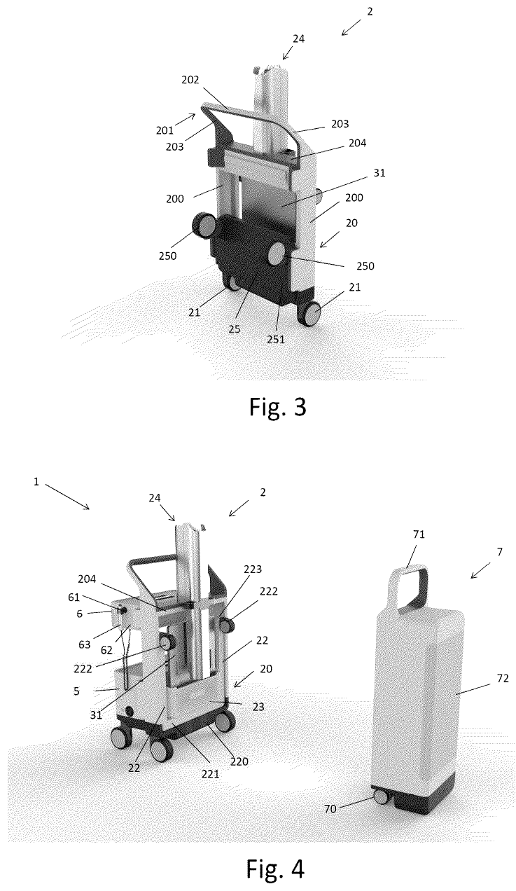

The medical imaging system 1 comprises a support 2. The support 2 is also shown individually in FIGS. 2 and 3.

The support 2 comprises a frame 20 or chassis. The frame or chassis constitutes a device enabling elements to be assembled. The frame 20 comprises two uprights 200. The uprights 200 are preferably parallel to each other.

The frame 20 here also comprises a gripping portion 201. The gripping portion 201 connects the uprights 200. The gripping portion 201 comprises a bar 202 continued on either side by two connecting portions 203. Preferably, the bar 202 is substantially orthogonal to the uprights 200. The connecting portions 203 are substantially orthogonal to the bar 202. Each connecting portion 203 continues one of the uprights 202.

The frame 20 is preferably made of metal.

The support 2 comprises at least two wheels 21. The wheels 21 are assembled to the frame 20. For example, a wheel 21 is fixed to one end of each upright 200. The wheels 21 allow the support 2 to be moved.

According to an exemplary embodiment, the wheels 21 can be removably assembled to the frame 20. For example, the wheels 21 can be fixed to a base, itself removably assembled to the frame 20.

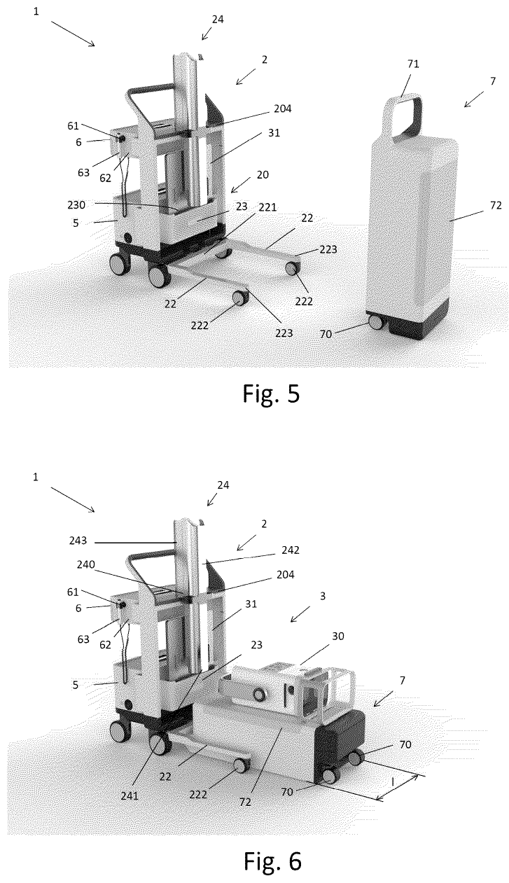

The support 2 further comprises two longitudinal members 22 (more clearly visible in FIGS. 4 and 5 for example). The longitudinal members 22 are assembled to the frame 20. The longitudinal members 22 are pivotably mounted on the frame 20. The longitudinal members 22 are configured to pivot relative to the frame between an operational position and a stowage position.

The frame 20 and the longitudinal members 22 are connected by a hinge 220. In the example shown, the support 2 comprises a connecting beam 221 extending orthogonally from the longitudinal members 22. The connecting beam 221 is integral with the two longitudinal members 22 and is connected to the frame 20 by the hinge 220.

FIGS. 1 to 4 show the longitudinal members 22 in the stowage position. In the stowage position, the longitudinal members 22 are parallel to the uprights 200. The longitudinal members 22 extend between the uprights 200. Each longitudinal member 22 is opposite one of the uprights 200. This configuration allows the support 2 to have greater compactness.

FIGS. 5 to 7 show the longitudinal members 22 in the operational position. In the operational position, the longitudinal members 22 extend substantially horizontally. The longitudinal members 22 extend orthogonally to the uprights 200.

Each longitudinal member comprises a wheel 222 at a free end 223. The wheels 222 facilitate the movement of the support 2 when the longitudinal members 22 are in the operational position. The support 2 can be pulled or pushed, irrespective of the position in which the longitudinal members 22 are located (operational position or stowage position).

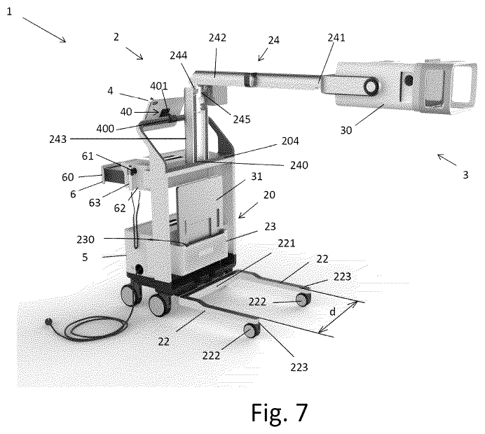

The medical imaging system 1 further comprises a medical imaging device 3 (visible in its entirety in FIGS. 6 and 7 and in part in the other figures). Preferably, the medical imaging device 3 is an X-ray device. The medical imaging device 3 here comprises an X-ray source 30. This source 30 operates in association with an X-ray detector 31. Preferably, the X-ray source 30 has an upper tube load limit of greater than or equal to 40 mAs, which then allows all the desired examinations to be carried out.

However, the medical imaging device 3 may be different, for example a tomography or echography device.

In the exemplary embodiment shown, the support 2 comprises a receiving tray 23. The receiving tray 23 comprises a seat 230 configured to receive the detector 31. The receiving tray 23 here has a parallelepipedal shape. The receiving tray 23 is fixed to the uprights 200 of the frame 20. Alternatively, the receiving tray 23 can be formed integrally with the uprights of the frame 20.

The receiving tray 23 preferably extends between the uprights 200 of the frame 20. This configuration also allows the support 2 to have greater compactness.

The support 2 also comprises an arm 24. The arm 24 is assembled to the frame 20. The arm 24 comprises a fixed end 240, assembled to the frame 20, and a free end 241. The fixed end 240 can be fixed to a fixing bar 204 of the frame 20 extending orthogonally to the uprights 200. The free end 241 is configured to receive the X-ray source 30.

The arm 24 comprises a front section 242 and a rear section 243. The rear section 243 is fixed to the fixing bar 204 by the fixed end 240. The front section 242 comprises the free end 241.

The front section 242 and the rear section 243 are articulated with respect to each other. The front section 242 and the rear section 243 are articulated about a pivot axis 244 (clearly visible in FIG. 7). The arm 24 is movable between a service position and a retracted position. The front section 242 and the rear section 243 are folded onto each other when the arm 24 is in the retracted position, as can be seen in FIGS. 1 to 6. The front section 242 and the rear section 243 together form a non-zero angle when the arm 24 is in the service position, preferably greater than a predefined angle (for example greater than an angle of 30°). For example, as can be seen in FIG. 7, the angle formed between the front section 242 and the rear section 243 is substantially a right angle.

The arm 24 can be movable between the service position and the retracted position manually or automatically. For example, the medical imaging system 1 further comprises an actuator 245 assembled to the arm 24. The actuator 245 is configured to move the arm 24 between the service position and the retracted position. The actuator 245 makes it possible to position the arm 24 at the desired height for carrying out the medical examination. The actuator 245 is preferably an electric actuator.

By virtue of the arm 24, the X-ray source 31 is assembled to the support in a removable manner. The assembly is carried out at the free end 241 of the arm 24. The removable assembly can be effected by any means of removable fixing, typically clipping, elastic interlocking, bolting, etc.

The free end 241 advantageously comprises a secondary alignment system permitting, with a primary alignment system for the X-ray source 31, an optimized alignment of the X-ray source 31 with the target (namely a patient and the detector 30). For example, the primary alignment system and the secondary alignment system are magnetic.

The use of an actuator limits the efforts by the manipulator/medical personnel. In fact, they do not need to lift the arm 24 with the X-ray source 30 in order to position it in the service position.

The medical imaging system 1 further comprises a control device 4. The control device 4 is configured to control the medical imaging device 3.

Preferably, the control device 4 is also configured to read and display an image received from the medical imaging device 3.

The control device 4 typically comprises a computer tool for data and image management. The control device 4 can comprise or be a tablet, in particular a touch-sensitive tablet (as in the example shown) or a computer. The control device 4 comprises in particular a display for displaying the received image.

The control device 4 is removably assembled to the support 2. The control device 4 can be assembled by any removable means, such as clipping, elastic interlocking, bolting, etc. The control device 4 is here assembled to the gripping portion 201 of the frame 20. In particular, the control device 4 is assembled to the bar 202. The control device 4 is here assembled to a fixing piece 40 (visible in FIG. 7), which is itself assembled to the bar 202. The fixing piece 40 comprises an attachment portion 400 in the form of a hook shaped to match the bar 202. The attachment portion 400 is elastic. The fixing piece 40 further comprises a support portion 401 continuing the attachment portion 400. The support portion 401 is configured to allow the control device 4, here the tablet, to be assembled. The control device 4 can, for example, be clipped or magnetically assembled to the support portion 401.

The control device 4 can also be joined to (or arranged on) a housing 6 described below, without using a fixing piece.

The medical imaging system 1 also comprises an electric power supply device 5. The electric power supply device preferably comprises at least one battery. The electric power supply device 5 is configured to supply electrical energy to electrical components of the medical imaging system 1, for example the medical imaging device 3 and/or the control device 4. The electric power supply device 5 can supply all or some of the electrical components of the medical imaging system 1.

The electric power supply device 5 is removably assembled to the support 2.

In the example shown, the support 2 comprises a receiving plate 25 (clearly visible in FIG. 2). The receiving plate 25 is configured to receive the electric power supply device 5. For this purpose, the receiving plate 25 has dimensions and a shape similar to those of the electric power supply device 5.

The receiving plate 25 is assembled to the frame 20, for example by a hinge connection. The receiving plate 25 is configured to pivot between an operational position and a stowage position.

FIGS. 1, 2 and 4 to 7 show the receiving plate 25 in the operational position. The receiving plate 25 extends substantially horizontally. The receiving plate 25 is orthogonal to the uprights 200 of the frame 20. The receiving plate 25 in the operational position is capable of receiving the battery 5.

FIG. 3 shows the receiving plate 25 in the stowage position. The receiving plate 25 extends substantially vertically. The receiving plate 25 is orthogonal to the uprights 200 of the frame 20.

The receiving plate 25 allows the battery 5 to be removably assembled to or mounted on the support 2.

The receiving plate 25 preferably comprises at least one wheel 250. The receiving plate 25 here comprises two wheels 250. The wheels 250 are fixed to an underside 251 of the receiving plate 25. The wheels 250 facilitate the movement of the support 2 when the receiving plate 25 is in the operational position. The support 2 can be pulled or pushed, irrespective of the position of the receiving plate 25 (operational position or stowage position).

In the exemplary embodiment where the wheels 21 are removably assembled to the frame 20 (for example by means of a base, itself removably assembled to the frame 20), the electric power supply device 5 is assembled to the frame 20 by a removable assembly system (e.g. hooks, clips, or any other means of mechanical or magnetic assembly). The removable assembly system is itself fixed to the frame 20, for example to the fixing bar 204. This configuration has the advantage of having a lower overall mass and of affording a medical imaging system with fewer wheels. For example, the medical imaging system can have just four wheels in total instead of six wheels as in the exemplary embodiment shown in the figures.

The medical imaging system 1 can also comprise a housing 6. The housing 6 is configured to be assembled to the support 2 in a removable manner.

The housing 6 is here clipped onto the fixing bar 204. The housing 6 can be assembled to the support 2 in other ways, for example by elastic interlocking or by bolting.

The housing 6 can be used for storage purposes by the medical personnel using the medical imaging system 1. The housing 6 comprises, for example, a drawer 60 sliding on rails as can be seen in FIG. 7. The housing 6 here comprises an assembly support 61. The assembly support 61 is fixed to a side wall 62 of the housing 6. The assembly support 61 is configured to receive a remote control 63 for imaging. The assembly support 61 thus allows the remote control 63 to be stowed while keeping it easily accessible when needed.

Advantageously, the housing 6 has a shape and/or dimensions identical to those of the battery 5. If it is necessary to stow or transport the housing 6 and the battery 5, this makes it possible to stack them and thereby facilitate stowage/transport.

The housing 6 can also serve as a support for the control device 4.

The medical imaging system 1 advantageously also comprises a case 7 shown in FIGS. 4 to 6. The case 7 is dimensioned to house at least the X-ray source 30 and/or the detector 31. The case can comprise at least one seat for accommodating the X-ray source 30 and/or the detector 31. The case 7 can also accommodate the control device 4. For this purpose, the case 7 can then comprise a specific seat for the control device 4. The case 7 can also contain other elements that may prove useful for carrying out the medical imaging examination.

Preferably, the case 7 has a width I substantially equal to a distance d between the longitudinal members 22 of the support 2. In the example shown, the longitudinal members 22 are parallel to each other. As can be seen in FIG. 6, this makes it possible to arrange the case 7 between the longitudinal members 22 when the longitudinal members 22 are in the operational position. The case 7 thus dimensioned is wedged between the longitudinal members 22.

The case 7 is preferably provided with two wheels 70. The case 7 also preferably comprises a handle 71. The medical personnel or manipulator can thus easily pull and manipulate the case 7.

According to another alternative or complementary exemplary embodiment, the case 7 can comprise straps allowing it to be transported on the back. The case 7 can thus be transported like a backpack.

When transporting or stowing the medical imaging system 1, the various parts (support, battery, housing, etc.) of the latter can be stowed in a compact manner. The medical imaging device 3, the control device 4, the electric power supply device 5 are separated from the support 2. The housing 6 is also separated from the support 2 if the medical imaging system 1 comprises a housing. Each of the parts of the medical imaging system 1 can thus be handled individually for its stowage or transport. The medical imaging system 1 is dimensioned to allow it to be transported and stowed in a standard trunk of a car. During transport or stowage, the longitudinal members 22 and the receiving plate 25 are in the stowage position, and the arm 24 is in the retracted position. The medical imaging system 1 is thus in what is called a transport configuration.

In order to move within the same premises, for example from room to room in a hospital, the medical imaging system 1 can adopt a movement configuration visible in FIG. 4 according to a particular example. In this configuration, the control device 4, the battery 5 and the housing 6 are assembled to the support 2. The X-ray detector 31 is housed in the receiving tray 23. The X-ray source 30 is stowed in the case 7. The longitudinal members 22 are in the stowage position. The receiving plate 25 is in the operational position so as to be able to receive the battery 5. The movement configuration allows the manipulator/medical personnel to move easily by pushing the support 2 and pulling the case 7. A single manipulator can move the medical imaging system 1 easily and with little effort.

When the manipulator arrives at the medical examination site, especially near the patient, he is easily able to deploy the medical imaging system 1. The longitudinal members 22 are first moved into the operational position, as in FIG. 5. The case 7 is then disposed between the longitudinal members 22 as in FIG. 6. A cover 72 of the case 7 is opened and the X-ray source 30 is taken out. The cover 72 is optionally closed again, and then the X-ray source 30 is placed on the closed cover 72. FIG. 6 shows the closed cover 72 and the X-ray source 30 removed from the case 7 and placed on the cover 72. The arm 24, until then in the retracted position, is deployed, for example by means of the actuator 245, in order to move as far as the X-ray source 30. The arm 24 can be assembled automatically to the X-ray source 30. The arm 24 finally assumes the service position, for example the one shown in FIG. 7. The medical imaging system 1 is thus in what is called a deployed configuration. It should be noted that the case 7 has not been shown in FIG. 7, but it can be kept between the longitudinal members 22 if necessary in the deployed configuration (for example if items housed in the case are needed during a medical examination).

After deployment of the medical imaging system 1, the X-ray detector 31 is removed from the receiving tray 23 in order to be positioned behind the patient for capturing an image.

The manipulator can easily and effortlessly arrange the medical imaging system 1 in the different configurations (transport configuration, movement configuration and deployed configuration). All manipulations (stowage, transport, deployment, etc.) can be done by one person.

The medical imaging system according to the invention is modular, ergonomic, compact and of light weight, while at the same time allowing a wide range of medical examinations.

1. A medical imaging system comprising:
a support having a frame;
at least two wheels assembled directly or indirectly to the frame so as to allow said support to be moved;
a medical imaging device;
a control device configured to control the medical imaging device; and
an electric power supply device configured to supply electrical energy to the medical imaging device and/or the control device,
the medical imaging device, the control device and the electric power supply device each being configured to be removably assembled to the support,
the medical imaging system wherein the support further comprises two longitudinal members assembled to the frame and configured to pivot relative to the frame between an operational position and a stowage position, the medical imaging system further comprising an X-ray source, an X-ray detector and a case, the case being dimensioned to house the X-ray source and/or the X-ray detector, the case having a width substantially equal to the distance between the longitudinal members.

2. The medical imaging system according to claim 1, further comprising a housing configured to be removably assembled to the support.

3. The medical imaging system according to claim 1, wherein the wheels are removably assembled to the frame.

4. The medical imaging system according to claim 1, wherein the wheels are assembled to a base, said base being removably assembled to the frame.

5. The medical imaging system according to claim 1, wherein the frame comprises two uprights and a gripping portion connecting the uprights.

6. The medical imaging system according to claim 5, wherein the longitudinal members, in the operational position, extend substantially horizontally, the longitudinal members, in the stowage position, being parallel to the uprights of the frame and extending between said uprights.

7. The medical imaging system according to claim 6, wherein each longitudinal member comprises a wheel at a free end.

8. The medical imaging system according to claim 5, wherein the support further comprises a receiving plate configured to receive the electric power supply device, the receiving plate being assembled to the frame and configured to pivot relative to the frame between an operational position and a stowage position, the receiving plate, in the operational position, extending substantially horizontally, the receiving plate, in the stowage position, being orthogonal to the uprights of the frame and extending substantially vertically.

9. The medical imaging system according to claim 8, wherein the receiving plate comprises at least one wheel on an underside.

10. The medical imaging system according to claim 1, wherein the X-ray source has an upper tube load limit of greater than or equal to 40 mAs.

11. The medical imaging system according to claim 1, wherein the support comprises a seat configured to receive the X-ray detector.

12. The medical imaging system according to claim 1, wherein the support comprises an arm assembled to the frame and having a free end configured to receive the X-ray source.

13. The medical imaging system according to claim 12, wherein the arm comprises a front section and a rear section articulated with respect to each other, the arm being movable between a service position and a retracted position, the front section and the rear section together forming a non-zero angle, preferably greater than 30°, when the arm is in the service position, the front section and the rear section being folded onto each other when the arm is in the retracted position.

14. The medical imaging system according to claim 13, further comprising an actuator assembled to the arm and configured to move the arm between the service position and the retracted position, the actuator preferably being an electric actuator.
